(19) United States
(12) Patent Application Publication
(10) Pub. No.: US 2025/0180418 A1
(43) Pub. Date: Jun. 5, 2025

(54) SENSOR ARRANGEMENT

(71) Applicant: Resense GmbH, Klingenberg (DE)

(72) Inventor: Sebastian Matich, Bad Mergentheim (DE)

(73) Assignee: Resense GmbH, Klingenberg (DE)

(21) Appl. No.: 18/967,336

(22) Filed: Dec. 3, 2024

(30) Foreign Application Priority Data
Dec. 4, 2023 (DE) 102023133864.1

(51) Int. Cl. G01L 1/22

(52) U.S. Cl. CPC G01L 1/2293; G01L 1/2218

(57) ABSTRACT

Sensor arrangement (1) for measuring a force and/or a torque, including a plurality of sensor sections (11), each having a sensor (13), the sensor sections (11) being arranged around a first axis (3); and a plurality of connecting pieces (21), each of which connects two adjacent sensor sections (11) to one another, a connection (33) between a connecting piece (21) and a first sensor section of adjacent sensor sections being provided in a first axial region (23), and the connecting piece (21) extending into a second axial region (25), which is different than the first axial region (23), in a gap (17) between the adjacent sensor sections.

### BACKGROUND OF THE INVENTION

The disclosure relates to a sensor arrangement for measuring a force or a torque, and to a method for producing a sensor arrangement.

### PRIOR ART

The prior art discloses arrangements of sensors that can measure forces or torques. Particularly for applications in medical engineering, for example in telemanipulators for minimally invasive surgery, or in robotics, for example in industrial gripping systems, such arrangements of sensors can be used to measure or control forces or torques. For example, a sensor arrangement can be used to provide haptic feedback pertaining to a gripping or manipulation arm or an end effector provided thereon. For many applications, the smallest or cheapest force and torque sensors are advantageous.

However, known sensor arrangements have restrictions, in particular with regard to miniaturization of the sensor arrangements. Alternatively or in addition, known sensor arrangements, in particular when the size of the sensor arrangements is being gradually reduced, may have restrictions with regard to a measurement accuracy or a robustness of the sensor arrangement or may require a high level of complexity during production.

### SUMMARY OF THE INVENTION

The object of the disclosure is to specify a sensor arrangement, for measuring a force or a torque, that is improved compared to the prior art. In particular, a sensor arrangement should be specified that can be constructed so as to be particularly small, robust or cheap or has a high measurement accuracy. In addition, a method for producing a sensor arrangement is intended to be specified.

The object is achieved by way of a sensor arrangement for measuring a force or a torque as disclosed herein and by way of a method also as disclosed herein.

According to one aspect, a sensor arrangement for measuring a force or a torque is specified, in particular for measuring a force and a torque. The sensor arrangement comprises a plurality of sensor sections, each having a sensor, the sensor sections being arranged around a first axis. The sensor arrangement comprises a plurality of connecting pieces, each of which connects two adjacent sensor sections to one another, a connection between a connecting piece and a first sensor section of adjacent sensor sections being provided in a first axial region, and the connecting piece extending into a second axial region, which is different than the first axial region, in a gap between the adjacent sensor sections.

According to a further aspect, a method for producing a sensor arrangement according to embodiments described herein is specified. The method comprises providing the plurality of sensor sections connected by way of the connecting pieces, the sensor sections being arranged in one plane. The method comprises coiling the sensor sections to arrange the sensor sections around the first axis.

According to typical embodiments, the sensor arrangement comprises a plurality of sensor sections, each having a sensor. Each of the sensor sections typically comprises a measuring body for receiving or transmitting forces or torques. Typically, the sensors of the sensor sections are arranged in or on the respective measuring bodies in such a way that measurements of the sensors are suitable for determining the forces or torques acting on the sensor arrangement. Typically, the sensors are flat or planar and arranged in particular flat on the measuring body.

In typical embodiments, the sensors of the sensor sections are measuring elements or in particular expansion measuring elements. Each of the expansion measuring elements is typically configured to determine an expansion or a mechanical strain of a respective sensor section, in particular of a measuring body of the sensor section. The expansion measuring element can comprise, for example, a film strain gauge (strain gauge) or a semiconductor strain gauge. In particular, a semiconductor strain gauge can be a silicon strain gauge (Si strain gauge). Si strain gauges, for example, can have a particularly small physical size. The sensor of a sensor section can comprise at least one expansion measuring element, in particular exactly one expansion measuring element or exactly two expansion measuring elements. The sensors are typically firmly connected, for example glued, soldered or joined by means of glass solder, to the respective sensor sections, in particular the respective measuring bodies.

Typically, the sensor sections are in at least substantially similar form, in particular in mechanically at least substantially similar form. “At least substantially mechanically similar” is intended to be understood to mean in particular that the mechanical properties and the shape of the sensor sections are at least substantially similar. However, the sensor sections can, for example, have differences in an electrical interconnection of the sensors, for example in a number of conductor tracks running through the sensor section. The plurality of sensor sections can also be referred to herein as a plurality of elementary cells. The forces or torques to be determined using the sensor arrangement can be determined on the basis of a plurality of measurements of the sensors of the respective sensor sections, or elementary cells.

In typical embodiments, the sensor sections are arranged around a first axis. The terms “axially”, “radially” and “in the circumferential direction” herein typically relate to the first axis. Typically, at least three sensor sections are arranged around the first axis, in particular at least four or at least 5, or at most 24, in particular at most 20 or at most 16. In typical embodiments, the sensor sections are arranged all around the first axis. For example, 3 sensor sections may be arranged all around the first axis, in particular as a tripod. In further examples, 6, 8 or 12 sensor sections may be arranged all around the first axis. For example, a sensor arrangement having 6 sensor sections may be arranged as a hexagonal structure, in particular as a hexapod structure. In further embodiments, the sensor sections may be arranged around a first axis in a screw or spiral arrangement or in a honeycomb structure. In particular, a sensor arrangement having a honeycomb structure can comprise a multiplicity of sensor sections, in particular also more than 20 sensor sections.

In typical embodiments, the sensor arrangement comprises a plurality of connecting pieces. Typically, each of the connecting pieces connects two adjacent sensor sections of the plurality of sensor sections to one another. Typically, each of the connecting pieces is arranged in a gap between two adjacent sensor sections. In embodiments, the connecting pieces mechanically connect the sensor sections to one another in series. In particular, each pair of two adjacent sensor sections in the series are typically connected to one another by way of a connecting piece. In typical embodiments, the sensor sections connected in series comprise a beginning sensor section and an end sensor section, which are not directly connected to one another by a connecting piece. In embodiments, the sensor sections connected by way of the connecting pieces are arranged in a manner coiled around the first axis.

According to typical embodiments, the connecting pieces have a smaller thickness than the sensor sections. Typically, each of the connecting pieces has at most half the thickness of a sensor section, for example at most one third or at most one fifth of the thickness of a sensor section. Typically, thickness refers to a thickness in a radial direction.

In typical embodiments, the sensor arrangement comprises a flexible printed circuit board. The flexible printed circuit board typically comprises connecting piece regions that form the connecting pieces of the sensor arrangement. In embodiments, the flexible printed circuit board comprises sensor section regions, each of the sensor sections of the sensor arrangement comprising one of the sensor section regions of the flexible printed circuit board. Typically, each of the sensor section regions of the flexible printed circuit board is in the form of part of a sensor section. Typically, each of the sensor section regions is firmly connected to a measuring body of a sensor section. For example, a sensor section region of the flexible printed circuit board may be glued to the measuring body or soldered to a sensor that is firmly connected to the measuring body. Typically, the measuring body has a greater thickness than the flexible printed circuit board, in particular in the radial direction. For example, the measuring body may be at least twice as thick as the flexible printed circuit board, in particular at least three times or at least five times as thick. In typical embodiments, the flexible printed circuit board is a flex conductor, in particular a foil-based flex conductor. The flexible printed circuit board can have one or more layers of foils or conductor tracks. For example, the flexible printed circuit board can have two conductor layers. The thickness of the flexible printed circuit board may be less than 0.5 mm, in particular less than 0.3 mm or less than 0.2 mm, for example approximately 0.1 mm.

Typically, each of the sensor section regions of the flexible printed circuit board has a wiring zone for wiring the sensor of the respective sensor section. A wiring zone of a sensor section region can have, for example, conductor tracks or contact pads for electrical connection to the sensor of the sensor section. Typically, each of the connecting pieces has conductor tracks for electrically connecting the plurality of sensor sections. In typical embodiments, the sensor section regions and the connecting pieces are formed by exactly one flexible printed circuit board, in particular in such a way that the exactly one flexible printed circuit board interconnects all sensor sections of the sensor arrangement.

In typical embodiments, a connection is provided between a connecting piece and a first sensor section of two adjacent sensor sections in a first axial region extending in the direction of the first axis. The connection between the connecting piece and the first sensor section can be formed, for example, by the transition between the sensor section region of a flexible printed circuit board that is arranged on the measuring body of the first sensor section and the connecting piece arranged in a gap between the adjacent sensor sections. Typically, the connecting piece extends into a second axial region, which is different than the first axial region, in the gap between the adjacent sensor sections. Typically, a further connection between the connecting piece and a second sensor section of the adjacent sensor sections is provided in the first axial region. In further embodiments, the further connection may be provided in a further axial region, which is different than the first axial region. The further connection between the connecting piece and the second sensor section is typically in a form analogous to the connection between the connecting piece and the first sensor section. In typical embodiments, the extent of a connecting piece into the second axial region can advantageously lengthen the path of the flow of forces between adjacent sensor sections, which in particular can lead to increased flexibility of the connecting piece.

According to typical embodiments, an axial extent of the second axial region is greater than an axial extent of the first axial region. In embodiments, an axial extent of the second axial region is greater than an extent of the connecting piece in a direction perpendicular to the first axis. In typical embodiments, each of the connecting pieces extends in the axial direction over at least one quarter, in particular at least one third, of an axial length of a sensor section. In typical embodiments, each of the connecting pieces in the second axial region extends in the axial direction over at least one quarter of an axial length of a sensor section. In embodiments, the connection and the further connection between a connecting piece and the adjacent sensor sections are arranged at an axial end of a sensor section region of a flexible printed circuit board that is closer to the sensor of the sensor section. In further embodiments, the connection and the further connection are arranged at an axial end of the sensor section region of the flexible printed circuit board that is further away from the sensor of the sensor section.

In embodiments, each of the connecting pieces has an axially extending first longitudinal section and an axially extending second longitudinal section. Typically, the first longitudinal section and the second longitudinal section are arranged in the same axial region, in particular in the second axial region. Typically, the first longitudinal section and the second longitudinal section of a connecting piece each have a first end and a second end. In embodiments, each of the first ends is connected to one of the adjacent sensor sections. For example, a first end of a first longitudinal section may be connected to a first sensor section of the adjacent sensor sections, and a first end of the second longitudinal section may be connected to a second sensor section of the adjacent sensor sections. Typically, the second ends of the first longitudinal section and the second longitudinal section are connected to one another by way of a deflection section of the connecting piece. The deflection section can provide a deflection through at least 90°, in particular through at least 120° or through at least 150°. In typical embodiments, the deflection section provides a deflection of at least substantially 180°. In particular, the connecting piece may be substantially U-shaped, the first longitudinal section and the second longitudinal section forming the legs of the U-shape. In embodiments, the deflection section can, for example, be in the form of an arc-shaped section of the connecting piece, in particular in the form of an arc-shaped section between the second ends of the first and second longitudinal sections. In particular, the deflection section may be in the form of a 180° arc, for example in a U-shaped connecting piece. In further embodiments, the connecting piece can have a different shape, for example, a V-shape having the first and second longitudinal sections as legs, the deflection section providing a deflection less than 180°.

According to typical embodiments, the connecting pieces connect each pair of two adjacent sensor sections in an articulated manner. Typically, the connecting pieces are flexible. Typically, each of the connecting pieces is in the form of a solid joint having a joint axis parallel to the first axis. The solid joint may be formed as a flexure hinge.

In typical embodiments, the first longitudinal section and the second longitudinal section are twisted. In particular, a surface orientation of a twisted longitudinal section changes from the first end of the longitudinal section to the second end of the longitudinal section. The first longitudinal section and the second longitudinal section may be twisted in a diametrically opposed manner in particular along an axial direction. The first longitudinal section and the second longitudinal section may also be referred to herein as first and second torsion zones of the connecting piece. In embodiments, a flexibility of the connecting piece over a length of the longitudinal sections can advantageously be adjusted. Further, connecting pieces described herein can provide “resilience”, or flexibility, in the radial direction, which in particular can facilitate assembly of the sensor arrangement.

Typically, the sensor sections are much more rigid than the connecting pieces. Arrangement of the sensor sections around the first axis, for example as a result of the sensor sections and the connecting pieces according to embodiments described herein being coiled, results in adjacent sensor sections typically being arranged at an angle to one another. The connecting pieces typically provide the connection by way of the angle between the adjacent sensor sections. In contrast to a connecting piece that extends only in the circumferential direction, which would be bent through the angle, the connecting pieces according to embodiments described herein can provide a higher level of flexibility. In particular, the connection by way of the angle between the adjacent sensor sections can be provided substantially by a torsion of the first and second longitudinal sections. For example, the first torsion zone and the second torsion zone can each provide a change of angle of approximately half the angle between the adjacent sensor sections. The connections of a connecting piece to the sensor sections or the deflection section typically experience only a low bending load around the joint axis of a connecting piece in the form of a solid joint. Typically, the deflection section of a connecting piece is substantially not twisted.

Embodiments described herein may have the advantage that increased flexibility of the connection between adjacent sensor sections is provided. In particular, the sensor sections can be arranged around the first axis in a smaller radius of curvature. For example, an outer diameter of the sensor arrangement, measured perpendicular to the first axis, can be reduced. In addition or alternatively, increased flexibility can provide greater mobility of the sensor sections during assembly of the sensor arrangement around the first axis or lower mechanical impact on the sensors.

In typical embodiments, the sensor arrangement comprises an electrical supply line. The electrical supply line typically comprises conductor tracks for operating the sensors of the sensor arrangement, in particular for supplying power or for interchanging data with the sensors. The electrical supply line typically extends in part outside an axial region of the sensor sections. In embodiments, the electrical supply line is arranged in the circumferential direction between two adjacent sensor sections. According to typical embodiments, the electrical supply line is arranged at one of the connecting pieces, typically at exactly one of the connecting pieces. In particular, the electrical supply line may be electrically directly connected to conductor tracks that run in the connecting piece at which the electrical supply line is arranged. For example, in embodiments in which the connecting piece and the electrical supply line are in the form of regions of a flexible printed circuit board, conductor tracks of the flexible printed circuit board run continuously from the electrical supply line into the connecting piece and in particular on to the sensor section regions. Typically, the electrical supply line runs substantially in the axial direction, in particular in an axial region of the sensor sections. In typical embodiments, the electrical supply line is in the form of part of a flexible printed circuit board of the sensor arrangement, in particular in the form of part of a flexible printed circuit board that forms connecting pieces and sensor section regions.

In embodiments, the electrical supply line is arranged at a deflection section of one of the connecting pieces, for example at an arc-shaped section of the connecting piece. In particular, the electrical supply line and the deflection section may be arranged in a substantially Y-shaped manner, the electrical supply line corresponding to the lower branch of the Y and the two upper branches of the Y corresponding to the deflection section, for example to a 180° arc-shaped deflection section. In embodiments, an arrangement at a deflection section may have the advantage that the flexibility of the connecting piece at which the electrical supply line is arranged is not impaired by the electrical supply line. In particular, in typical embodiments, the deflection section is not bent or twisted, and so a flexibility resulting from a torsion of the first and second longitudinal sections remains unaffected.

In typical sensor arrangements, the connecting pieces mechanically connect the sensor sections in series according to embodiments described herein. Typically, the electrical supply line is provided at a connecting piece that is arranged centrally in the series, in particular centrally in the series between a beginning sensor section and an end sensor section, which are not directly connected by a connecting piece. A centrally arranged connecting piece is understood to mean the central connecting piece in the series when the number of sensor sections is even. When the number of sensor sections is uneven, the electrical supply line may be provided at one of the two connecting pieces arranged centrally in the series. A central arrangement of the electrical supply line can in particular reduce the number of conductor tracks in the individual connecting pieces. For example, the conductor tracks to the sensor sections located on the beginning sensor section side can be routed via the first longitudinal section of the centrally arranged connecting piece, and the conductor tracks to the sensor sections located on the end sensor section side can be routed via the second longitudinal section of the centrally arranged connecting piece. For example, the number of conductor tracks through individual connecting pieces can be halved. Reducing the number of conductor tracks in a connecting piece makes the connecting piece more flexible. Furthermore, a connecting piece can be made narrower when the number of conductor tracks is reduced, which can in particular again increase the flexibility of the connecting piece.

In further embodiments, the electrical supply line may be arranged at another of the connecting pieces. In still further embodiments, the electrical supply line may be arranged at the beginning sensor section or the end sensor section. In particular, an electrical supply line can have a supply line connecting piece for connecting the electrical supply line to the beginning sensor section or the end sensor section. The supply line connecting piece can have a deflection section and one or two longitudinal sections, like connecting pieces described herein. For example, the supply line connecting piece may be substantially U-shaped. The supply line connecting piece may be arranged in a gap between the beginning sensor section or the end sensor section. The supply line connecting piece can provide a higher level of flexibility of the electrical supply line, for example in order to reduce mechanical impact on the beginning sensor section or the end sensor section.

In typical embodiments, the sensor arrangement comprises two covers, in particular a first cover and a second cover, wherein each of the sensor sections is arranged at least in part axially between the first cover and the second cover. The covers can, for example, be in the form of disks, in particular in the form of disks arranged coaxially with the first axis. The disks can, for example, be substantially circular. In typical embodiments, the covers or the measuring bodies of the sensor sections may be made from metal, for example.

Typically, one of the covers has a supply line recess, in particular for routing the electrical supply line in the axial direction. Typically, the supply line recess is provided in a radially outer surface of the cover, for example as a slot or groove. In further embodiments, the supply line recess may be in the form of an axial opening or through-opening in the cover. In typical embodiments, the supply line recess is provided in the cover between two sensor sections that are adjacent in the circumferential direction. The electrical supply line is typically arranged in the supply line recess, in particular arranged in the axial direction through the supply line recess. The arrangement of the electrical supply line and the supply line recess in the circumferential direction between adjacent sensor sections may have the advantage that the cover is not weakened in a region of the sensor sections by the supply line recess, in particular in embodiments in which the cover has further openings or recesses for receiving spigots of the sensor sections. In particular, a robustness of the sensor arrangement can be increased. Alternatively, bending of the electrical supply line can be avoided compared to a version without a supply line recess. Further, embodiments may have the advantage that the electrical supply line and the supply line recess are arranged away from a weld seam between the spigot and the cover, which in particular facilitates assembly of the sensor arrangement.

In illustrative embodiments, 6 sensor sections can be arranged hexagonally around the first axis between two covers, in particular to form a hexapod. For example, the sensor arrangement can be used to measure three different force components or three different torque components independently of one another, in particular three different force components and three different torque components.

According to typical embodiments, each of the sensor sections comprises a measuring body. The measuring body can, for example, be substantially cuboid. In particular, the structure of coiled sensor sections can substantially correspond to a polygon. Typically, the measuring body has the greatest extent in the axial direction. Typically, the measuring body comprises a weakened region. Typically, the measuring body is tapered in the weakened region, in particular in a region of the sensor of the sensor section. The weakened region can have at least one weakening recess, in particular two weakening recesses. A weakening recess can, for example, be in the form of an opening, a hole or a cutout in the measuring body, e.g. in the form of a circular hole or in the form of an L-shaped or C-shaped cutout. Typically, the measuring body comprises a first side facing the first axis. In typical embodiments, the at least one weakening recess is provided at least substantially perpendicularly through the first side.

Typically, the measuring body has, in the weakened region, a bridge that connects parts of the measuring body between a first axial end and a second axial end of the weakened region to one another. In particular, the bridge can run between two weakening recesses. The weakening recesses may be arranged such that the bridge running between the weakening recesses forms an angle with an axial direction, for example an angle of at least 30°, in particular of at least 35° or of at least 40°, or of at most 60°, in particular of at most 55°.

In embodiments, the measuring bodies or the bridges of the sensor sections can be arranged in a manner inclined with respect to one another. For example, in a sensor arrangement having 6 sensor sections, the bridges of the measuring body can be arranged hexagonally around the first axis. The bridges can be arranged in an inclined manner, in particular in a manner inclined relative to one another, between two covers, in particular to form a hexapod.

Typically, the sensor of a sensor section is arranged in the weakened region of the sensor section, in particular on the bridge of the weakened region. In the weakened region, in particular expansions of the measuring body can be measured precisely by the sensor. In embodiments, the measuring body typically comprises a substantially rigid receiving region for receiving the sensor section region of the flexible printed circuit board. The receiving region may be provided in a manner axially offset from the weakened region. Typically, the wiring zone of a sensor section region of a flexible printed circuit board is arranged on the receiving region of the measuring body. Typically, the connecting pieces extend at least substantially within the same axial region as the receiving region of the measuring body.

In typical embodiments, a sensor section comprises a sensor supply line, in particular a sensor supply line for electrical connection between the sensor and a sensor section region of a flexible printed circuit board. In embodiments, the sensor supply line can be provided by bonding wires between the sensor and the sensor section region. For example, the bonding wires can be electrically connected to contact pads of the sensor section region. In further embodiments, the sensor supply line can be in the form of a sensor supply line region of the flexible printed circuit board. For example, a sensor supply line region can be connected to a sensor by way of one or more solder points. The sensor supply line region can be in the form of a sensor supply line connecting piece having multiple arcs or can be meandrous, in particular in order to provide a high level of flexibility or low mechanical action between the sensor section region of the flexible printed circuit board and the sensor.

In typical embodiments, each of the sensor sections, in particular the measuring bodies of the sensor sections, comprises at least one spigot at an axial end of the respective sensor section. Typically, each of the sensor sections comprises two spigots, in particular one spigot at each of the two axial ends of a sensor section. Typically, the spigots are configured to engage in the first cover or the second cover of the sensor arrangement. The first cover or the second cover can have corresponding openings or recesses to receive the spigots of the sensor sections. A firm connection between the spigot and the cover can be provided, for example, by a plug connection between the spigot and the cover or by a weld between the spigot and the cover, in particular by a plug connection and a weld.

In typical embodiments, the sensor arrangement has a diameter perpendicular to the first axis of not more than 15 mm, in particular of not more than 10 mm or not more than 8 mm. For example, the sensor arrangement can have a diameter of approximately 8 mm or approximately 6 mm. The diameter refers to an outer diameter. In other embodiments, the sensor arrangement has a diameter perpendicular to the first axis of not more than 21° mm or 32° mm. Embodiments described herein provide, for example, increased flexibility of the connecting pieces or increased robustness of the connection of the cover and the sensor sections, in particular for miniaturizing sensor arrangements described herein for force or torque measurement. In typical embodiments, an axial length of the sensor arrangement is less than 20 mm, in particular less than 15 mm, in particular without taking into account an axial extent of an electrical supply line.

According to typical embodiments, a method for producing a sensor arrangement is specified, in particular a sensor arrangement according to embodiments described herein. The method comprises providing the plurality of sensor sections connected by way of the connecting pieces. Typically, the sensor sections are arranged in one plane, in particular in a plane parallel to a first axis. Typically, provision of the sensor sections and connecting pieces comprises providing measuring bodies according to embodiments described herein, in particular each having a sensor that is arranged on or in the measuring body. A flexible printed circuit board can be provided, having sensor section regions and connecting pieces arranged between the sensor section regions according to embodiments described herein. The flexible printed circuit board can be arranged in the plane. The measuring bodies can be firmly connected to respective sensor section regions of the flexible printed circuit board. The sensors can be electrically connected to the respective sensor section regions. The parallel arrangement of the sensor sections in one plane allows these production steps to be carried out cheaply, for example.

Typically, the method comprises coiling the sensor sections to arrange the sensor sections around the first axis. When coiling the sensor sections, the sensor sections can be arranged at an angle to one another, in particular the connecting pieces serving as solid joints between adjacent sensor sections. Typically, the connecting pieces are not twisted before being coiled. Typically, the connecting pieces, in particular a first longitudinal section and a second longitudinal section of each of the connecting pieces, are twisted while being coiled.

In typical embodiments, the method comprises connecting the sensor sections to at least one cover, in particular to a first cover and a second cover. In particular, the sensor sections and the at least one cover can be firmly connected to one another. The at least one cover can be provided according to embodiments described herein. In embodiments, the sensor sections, in particular the coiled sensor sections, are arranged axially between the first cover and the second cover. The sensor sections can have a spigot at each of the axial ends of the sensor sections. The spigots can be engaged with openings or recesses in the first cover and the second cover. In addition or alternatively, the sensor sections and the at least one cover can be materially bonded, in particular glued to one another or welded. An electrical supply line can be arranged in a supply line recess in the first cover or the second cover.

Compared to the state of the art, typical sensor arrangements can afford the advantage that sensor arrangements having a smaller outer diameter can be produced. In particular, increased flexibility of the connecting pieces can be provided, as a result of which, for example, small radii of curvature in the circumferential direction between the sensor sections can be used. Typical sensor arrangements can have low mechanical impact on the strain gauge sensor system, in particular as a result of twisting of the torsion zones of the connecting pieces. Intrinsic stresses in the flexible printed circuit board, which have an adverse effect on the measurement accuracy of the sensor arrangement, can be reduced. In particular, additional, interfering forces can be avoided that would be coupled into the sensor sections fitted with sensors if the connecting pieces were less flexible. Embodiments can have an improved drift behavior, in particular can avoid or reduce a nonlinear or nonreproducible temperature response. Further, embodiments can afford the advantage that great mobility of the sensor sections can be provided during assembly. Typical embodiments can further have improved signal removal. In particular, the electrical supply line according to embodiments may be harmless for a robustness of the connection between the sensor sections and the cover. Further, the electrical supply line can have an advantageous effect in terms of flexibility of the connecting pieces.

### BRIEF DESCRIPTION OF THE DRAWINGS

Further advantages and features of preferred embodiments of the invention will be explained below on the basis of the appended drawings, in which:

FIG. 1 shows a view of a sensor arrangement according to a typical embodiment;

FIG. 2 shows a schematic view of a plurality of sensor sections connected to one another by way of connecting pieces, according to the embodiment of FIG. 1;

FIG. 3 shows a detail of the flexible printed circuit board according to the sensor arrangement of FIG. 1, in a perspective view from the first cover axially in the direction of the second cover;

FIGS. 4 to 6 each show a schematic view of a plurality of sensor sections and connecting pieces according to further typical embodiments;

FIG. 7 shows a flowchart for a method for producing a sensor arrangement according to a typical embodiment.

### DETAILED DESCRIPTION

Typical embodiments will be described below on the basis of the figures, wherein the invention is not restricted to the exemplary embodiments; instead the scope of the invention is determined by the claims.

In the description of the figures, the same reference signs are used for identical or similar parts. In some instances, features which have already been described in connection with other figures are not described again for the sake of clarity.

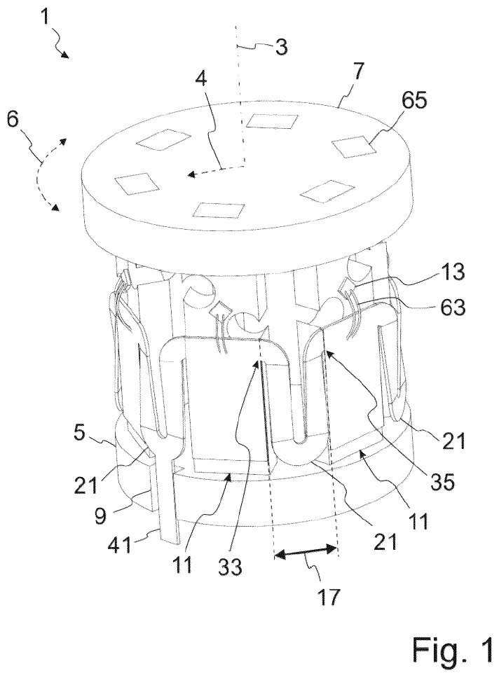

FIG. 1 shows a schematic view of a sensor arrangement 1 according to typical embodiments. The sensor arrangement 1 is in particular in the form of a hexapod having 6 sensor sections 11. The sensor sections 11 are mechanically connected to one another in series by way of connecting pieces 21. The sensor sections 11 are arranged, in particular coiled, all around a first axis 3. Adjacent sensor sections 11 in the series are arranged at an angle of 60° to one another in FIG. 1. The connecting pieces 21 arranged in the gaps 17 between the sensor sections 11 serve as solid joints between the sensor sections 11. The sensor sections 11 are arranged axially in part between a first cover 5 and a second cover 7 and firmly connected to the first cover 5 and the second cover 7. In particular, spigots 65, which are arranged at each of the axial ends of the sensor sections 11, engage in corresponding openings in the first cover 5 and the second cover 7. The thickness of the connecting pieces 21 in the direction perpendicular to the axis 3, in particular in the radial direction 4, is less than one third of the thickness of the sensor sections 11.

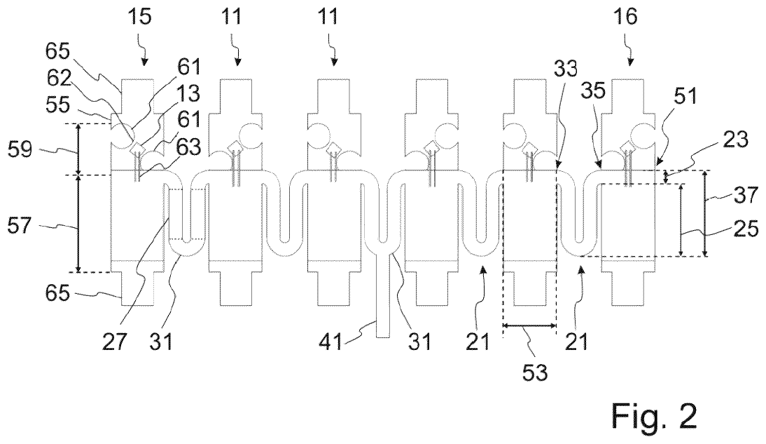

FIG. 2 shows the sensor sections 11 and connecting pieces 21 according to FIG. 1 in an uncoiled state, in particular without the first cover 5 and the second cover 7. Each of the sensor sections 11 comprises a measuring body 55, a sensor 13 and a sensor section region 53 of a flexible printed circuit board 51, the sensor section region 53 being firmly connected to the measuring body 55. The measuring body 55 has the two spigots 65. The measuring body 55 of a sensor section 11 comprises a weakened region 59, in which weakening recesses 61, in FIG. 1 two holes through the measuring body 55, are arranged around a bridge 62 of the measuring body 55. The sensor 13, in FIGS. 1, 2 and 4 an Si strain gauge, is permanently arranged on the bridge 62. Further, the measuring body 55 comprises a relatively rigid receiving region 57, axially adjoining the weakened region 59, that is firmly connected to the sensor section region 53 of the flexible printed circuit board 51. The sensor 13 and the sensor section region 53 are electrically connected to one another by way of a sensor supply line 63, in FIG. 1 bonding wires.

The flexible printed circuit board 51 further comprises the connecting pieces 21 and an electrical supply line 41 for operating the sensor arrangement 1, in particular the sensors 13. Each of the connecting pieces 21 is connected to two adjacent sensor sections 11. The sensor sections 11 mechanically connected in series comprise a beginning sensor section 15 and an end sensor section 16, which are not directly connected to one another by a connecting piece 21. Each of the connecting pieces 21 has a connection 33 to a first sensor section of two adjacent sensor sections, and a further connection 35 to a second sensor section of the adjacent sensor sections. In the embodiments with a flexible printed circuit board 51, the connection 33 and the further connection 35 are each formed by a transition by the flexible printed circuit board 51 from a sensor section region 53 to the connecting piece 21, in particular at an axial end of the sensor section region 53 that is closer to the sensor 13. The connection 33 and in particular also the further connection 35 are provided in a first axial region 23. Each of the connecting pieces 21 extends beyond the first axial region 23 into a second axial region 25, which is different than the first axial region 23, in the gap 17 between two adjacent sensor sections 11. An axial extent 37 of the connecting pieces 21 is greater than one third of an axial extent of the sensor sections 11.

Each of the connecting pieces 21 comprises a first longitudinal section 27 and a second longitudinal section 29, which extend in the same axial region, in particular the second axial region 25. In the uncoiled state of FIG. 2, the first and second longitudinal sections are arranged flat and in an axial orientation. The first longitudinal section 27 of a connecting piece 21 and the second longitudinal section 29 of the connecting piece 21 are connected to one another by way of a deflection section 31 of the connecting piece 21, in particular by a 180°arc-shaped deflection section 31. Overall, the connecting pieces 21 are substantially U-shaped.

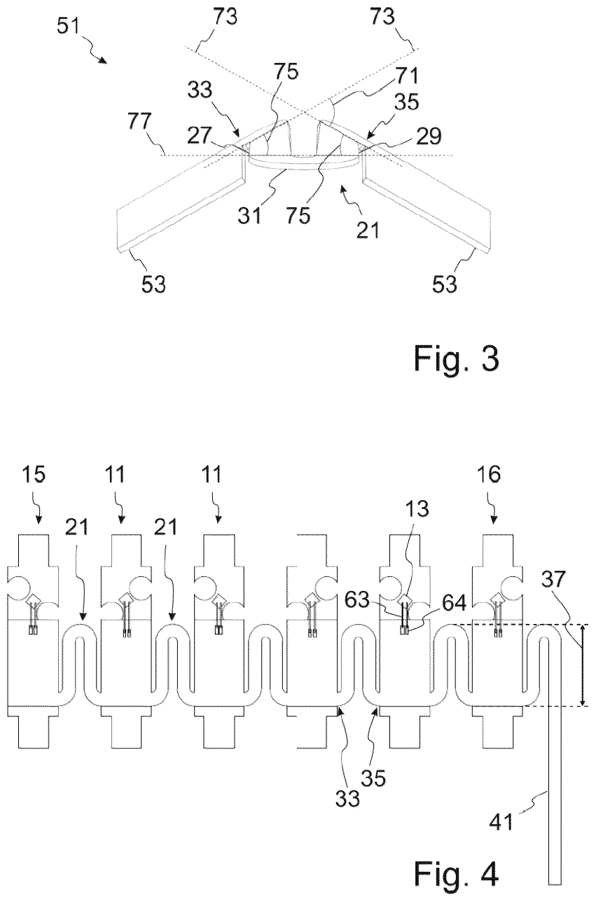

FIG. 3 shows a perspective view of a detail of the flexible printed circuit board 51 of FIG. 1, in particular from the first cover 5 in the direction of the second cover 7. In particular, FIG. 3 shows two sensor section regions 53 of the flexible printed circuit board 51, the two sensor section regions 53 being arranged at an angle 71 of 60° to one another. The two sensor section regions 53 are connected to one another by way of a connecting piece 21, the first longitudinal section 27 and the second longitudinal section 29 of the connecting piece 21 being twisted. The deflection section 31 remains substantially untwisted and unbent. As depicted schematically in FIG. 3, a line 77 along the surface of the longitudinal sections at the transition to the deflection section 31 forms the angles 75 with extension lines 73 of the sensor section regions 53, each of the angles 75 being approximately 30°. In particular, the angle 71 of 60° between two sensor sections is provided by diametrically opposed torsion through 30° of the first longitudinal section 27 and the second longitudinal section 29. The connecting piece 21 has a high level of flexibility, low bending load and low impact on the sensor system.

As shown in FIGS. 1 and 2, the flexible printed circuit board 51 comprises the electrical supply line 41, which is provided at a connecting piece 21 that is arranged centrally in the series of sensor sections 11, in particular in a Y-shape, at a deflection section 31 of the connecting piece 21. The central arrangement allows the number of conductor tracks per connecting piece to be reduced compared to a supply line at the end. The smaller number of conductor tracks also makes the connecting pieces more flexible. In FIG. 1, the first cover 5 further has a supply line recess 9 for axially routing the electrical supply line 41 to the connecting piece 21. The electrical supply line 41 and the supply line recess 9 are arranged in the circumferential direction 6 between two sensor sections. In particular, the supply line recess is arranged in the circumferential direction 6 in a manner offset from the recesses in the first cover 5 for the spigots 65 of the sensor sections 11, as a result of which in particular a robustness of the first cover 5 is increased.

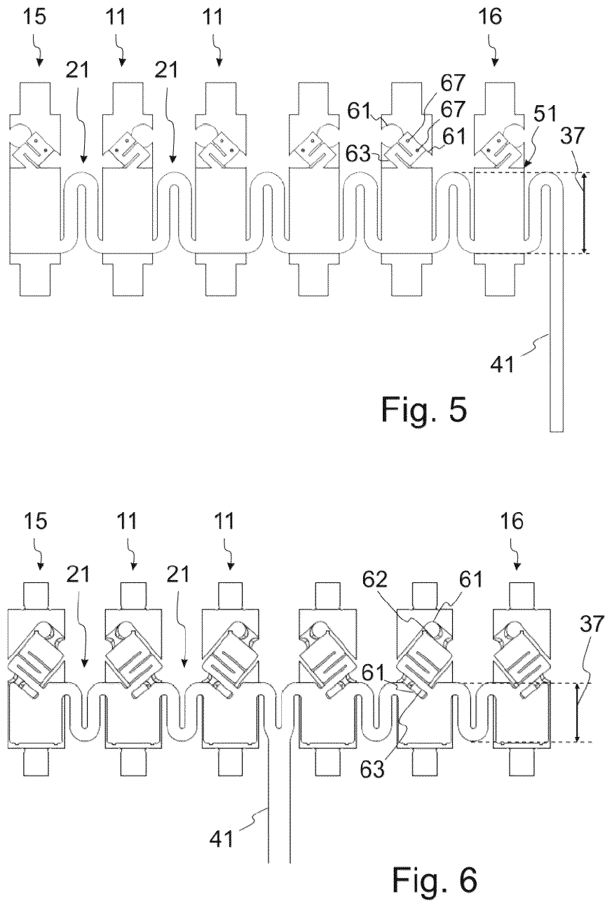

FIG. 4 shows a plurality of sensor sections 11 and connecting pieces 21 in the uncoiled state for a sensor arrangement according to a further embodiment. In FIG. 4, the connection 33 and the further connection 35 between the connecting pieces 21 and the sensor sections 11 are each provided at an axial end of the sensor section region 53 that is further away from the sensor 13. Further, the electrical supply line 41 is arranged at the end, in particular at the end sensor section 16. The electrical supply line 41 comprises a U-shaped supply line connecting piece, which is similar to the connecting pieces 21. Further, FIG. 4 shows, on each of the sensor section regions 53, for example two contact pads 64 configured for connection to the sensors 13 by way of bonding wires (sensor supply line 63). In other embodiments, for example three, four or five contact pads may also be present.

FIG. 5 shows a plurality of sensor sections 11 and connecting pieces 21 in the uncoiled state for a sensor arrangement according to yet another embodiment. The sensor supply line 63 in FIG. 5 is in the form of part of the flexible printed circuit board 51. In particular, each of the sensor sections 11 comprises a sensor supply line 63 that is in the form of a meandrous sensor supply line connecting piece between the sensor section region 53 of the sensor section 11 and the sensor (concealed by the sensor supply line 63 in FIGS. 5 and 6). For example, the sensors of FIGS. 5 and 6 are film strain gauges. An electrical connection between the sensor supply line 63 and the sensor is provided by way of solder points 67. Similarly to in FIG. 4, the electrical supply line 41 is arranged at the end.

FIG. 6 shows a plurality of sensor sections 11 and connecting pieces 21 in the uncoiled state for a sensor arrangement according to a further embodiment. Each of the sensor sections 11 in FIG. 6 has two weakening recesses 61, which are cut out in a substantially inclined manner in a C-shape around a bridge 62. An axial extent 37 of the connecting pieces 21 in FIG. 5 is, for example, approximately one third of the axial extent of the sensor sections 11. Similarly to in FIG. 2, the electrical supply line 41 is arranged at a central connecting piece 21 in the series of sensor sections 11.

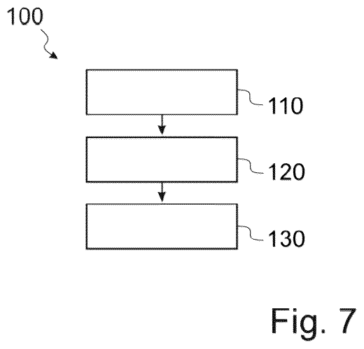

FIG. 7 shows a flowchart for a method 100 for producing a sensor arrangement 1 according to embodiments described herein. In block 110, the method 100 comprises providing the plurality of sensor sections 11 connected by way of the connecting pieces 21, the sensor sections 11 being arranged in one plane. In block 120, the method 100 comprises coiling the sensor sections 11 to arrange the sensor sections 11 around a first axis 3. In block 130, the method 100 comprises connecting the sensor sections 11 to a first cover 5 and a second cover 7. The high flexibility of the connecting pieces means that the production of the sensor arrangement and in particular the coiling of the sensor sections can be improved. For example, embodiments can result in detachment of the flexible printed circuit board from measuring bodies of the sensor sections being avoided or an impact of the connecting pieces on the sensors of the sensor sections being reduced.

1. Sensor arrangement (1) for measuring a force and/or a torque, comprising
a plurality of sensor sections (11), each having a sensor (13), the sensor sections (11) being arranged around a first axis (3); and
a plurality of connecting pieces (21), each of which connects two adjacent sensor sections (11) to one another, a connection (33) between a connecting piece (21) and a first sensor section of adjacent sensor sections being provided in a first axial region (23), and the connecting piece (21) extending into a second axial region (25), which is different than the first axial region (23), in a gap (17) between the adjacent sensor sections.

2. Sensor arrangement (1) according to claim 1, wherein each of the connecting pieces (21) has an axially extending first longitudinal section (27) and an axially extending second longitudinal section (29), and wherein the first longitudinal section (27) and the second longitudinal section (29) are arranged in the same axial region.

3. Sensor arrangement (1) according to claim 2, wherein the first longitudinal section (27) and the second longitudinal section (29) are twisted.

4. Sensor arrangement (1) according to claim 2, wherein the first longitudinal section (27) and the second longitudinal section (29) of a connecting piece (21) each have a first end and a second end, each of the first ends being connected to one of the adjacent sensor sections (11), and the second ends being connected to one another by way of a deflection section (31) of the connecting piece (21).

5. Sensor arrangement (1) according to claim 1, wherein a further connection (35) between the connecting piece (21) and a second sensor section of the adjacent sensor sections is provided in the first axial region (23).

6. Sensor arrangement (1) according to claim 1, wherein each of the connecting pieces (21) is in the form of a solid joint having a joint axis parallel to the first axis (3).

7. Sensor arrangement (1) according to claim 1, wherein each of the connecting pieces (21) extends in the axial direction over at least one quarter of an axial length of a sensor section (11).

8. Sensor arrangement (1) according to claim 1, wherein the sensor arrangement (1) further comprises an electrical supply line (41), the electrical supply line (41) being arranged at one of the connecting pieces (21).

9. Sensor arrangement (1) according to claim 8, wherein the electrical supply line (41) is arranged at a deflection section (31) of one of the connecting pieces (21).

10. Sensor arrangement (1) according to claim 8, wherein the connecting pieces mechanically connect the sensor sections in series, and wherein the electrical supply line (41) is provided at a connecting piece that is arranged centrally in the series.

11. Sensor arrangement (1) according to claim 1, further comprising a flexible printed circuit board (51) having sensor section regions (53) and connecting piece regions, wherein each of the sensor sections (11) comprises one of the sensor section regions (53) of the flexible printed circuit board (51), and wherein the connecting piece regions of the flexible printed circuit board (51) provide the connecting pieces (21).

12. Sensor arrangement (1) according to claim 1, further comprising a first cover (5) and a second cover (7), wherein each of the sensor sections (11) is arranged at least in part axially between the first cover (5) and the second cover (7).

13. Sensor arrangement (1) according to claim 1, wherein the plurality of sensor sections (11) are arranged all around the first axis (3).

14. Sensor arrangement (1) according to claim 1, wherein the sensor arrangement (1) has a diameter perpendicular to the first axis (3) of not more than 15 mm.

15. Method (100) for producing a sensor arrangement (1) according to claim 1, comprising
providing the plurality of sensor sections (11) connected by way of the connecting pieces (21), the sensor sections (11) being arranged in one plane; and
coiling the sensor sections (11) to arrange the sensor sections (11) around the first axis (3).
